(19) United States
(12) Patent Application Publication
(10) Pub. No.: US 2023/0082652 A1
(43) Pub. Date: Mar. 16, 2023

(54) CAPILLARY ASSISTED VITRIFICATION PROCESSES AND MATERIALS FOR PRESERVATION OF BIOLOGICAL SAMPLES

(71) Applicant: SOMNIO GLOBAL HOLDINGS, LLC, Novi, MI (US)

(72) Inventors: Pravansu S. Mohanty, Ann Arbor, MI (US); Subhendu Das, Ann Arbor, MI (US); Laura Bronsart, Ann Arbor, MI (US)

(21) Appl. No.: 17/904,534

(22) PCT Filed: Feb. 26, 2021
(86) PCT No.: PCT/US2021/019887

§ 371 Date: Aug. 18, 2022

Related U.S. Application Data
(60) Provisional application No. 62/982,856, filed on Feb. 28, 2020.

(51) Int. Cl. A01N 1/02

(52) U.S. Cl. CPC A01N 1/0221; A01N 1/0284

(57) ABSTRACT

Disclosed is a process for storage of nucleic acids from a biological sample. The process comprises providing a biological sample comprising one or more cells containing nucleic acids therein; contacting the biological sample with a vitrification medium comprising a vitrification agent and a lysing agent to form a vitrification mixture; vitrifying the vitrification mixture to generate a storage-stable sample. In various aspects, the storage-stable sample can be stored at a temperature above cryogenic temperatures, such as at room temperature, for 20 days or more.

### CROSS REFERENCE TO RELATED APPLICATIONS

This application depends from and claims priority to U.S. Provisional Application No. 62/982,856 filed Feb. 28, 2020, the entire contents of which are incorporated herein by reference.

### FIELD

The present disclosure relates to preservation of biological samples, and in particular, vitrification of biological materials for preservation of or portions of blood, saliva or tissue samples.

### BACKGROUND

Vitrification is the process of direct transition from a liquid to an amorphous glassy state and is often utilized to preserve biological materials by cooling them to cryogenic temperatures at high cooling rates. At cryogenic temperatures, vitrification techniques avoid the damaging effects of ice crystals, which are known to form during conventional cryopreservation. However, in order to avoid ice-nucleation during cooling, extremely high and potentially toxic concentrations (6-8 M) of cryoprotectants (CPAs) are required. Among the most commonly used CPAs are dimethyl sulfoxide (DMSO), glycerol, ethylene glycol (EG) and 1,2-propanediol (PROH). As a result, multiple steps and complex elaborate protocols are required to load and unload CPAs into cells.

Anhydrous vitrification at ambient temperatures may be an alternative strategy for preserving biological materials. In nature, a wide variety of organisms can survive extreme dehydration that correlates in many cases with the accumulation of large amounts (as much as 20% of their dry weight) of glass forming sugars such as trehalose and sucrose in intracellular space. However, dry preservation suffers from a major limitation in long-term storage due to the degradation of the biological material by cumulative chemical stresses encountered as the vitrification solution gets concentrated in the extra-cellular space. This results in irreversible cell damage, including damage to proteins and nucleic acids before the cells and the vitrification solution can reach a suitably low moisture content to become glassy. Therefore, there exists a need for improved vitrification processes to vitrify biological materials by fast drying while preserving the proteins and nucleic acids.

### SUMMARY

The following summary is provided to facilitate an understanding of various aspects described herein and is not intended to be a full description. A full appreciation of the various aspects can be gained by taking the entire specification, claims, drawings, and abstract as a whole.

Various aspects herein provide a process for storage of proteins, specifically intracellular nucleic acids, or other biological materials from within a cell, blood, saliva, tissue or other sample of an organism and within a biological sample. The process comprises providing a biological sample including one or more cells containing nucleic acids therein; contacting the biological sample with a vitrification medium comprising a vitrification agent and a lysing agent to form a vitrification mixture; and vitrifying the vitrification mixture to generate a storage-stable sample. In various aspects, the storage-stable sample can be stored at a temperature above cryogenic temperatures, such as at room temperature or higher, and optionally for 20 days or more.

Also provided are processes of preserving tissue samples that provide structural support and do not introduce cell damaging materials and thereby more effectively preserve cellular structure for subsequent histology or other tissue studies. A process includes contacting a target biological tissue with a polymer, a vitrification agent, and optionally a crosslinking agent and/or energy suitable to link the polymer to one or more components of the extracellular structure of cells in the tissue; vitrifying the tissue so as to form a vitrified tissue sample, and optionally sectioning the tissue (prior to or following vitrification) into one or more thin strips of tissue. Processes optionally further include rehydrating the vitrified tissue with a releasing agent and or energy that releases the polymer from the tissue so that viable and well-preserved tissue is available for subsequent analyses.

### BRIEF DESCRIPTION OF THE DRAWINGS

The drawings are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention. Exemplary aspects will become more fully understood from the detailed description and the accompanying drawings, wherein:

FIG. 1 schematically illustrates an example vitrification method that can be used according one or more aspects shown and described herein;

FIG. 2 schematically illustrates another exemplary vitrification method illustrating a biological sample of blood cells and a vitrification medium within a membrane for vitrification according to one or more aspects shown and described herein;

FIG. 3 schematically illustrates example methods for extracting RNA from storage-stable samples generated according to one or more aspects shown and described herein;

FIG. 4 illustrates an exemplary system for isolation of desired material and optional separation from contaminants such as bacteria or other undesirable organisms;

FIG. 5 is an image of an electrophoresis gel showing the degradation of RNA extracted from Comparative Examples A-C, using conventional nucleic acid storage techniques, and for Example 1, using processes of storing nucleic acids according to one or more aspects shown and described herein;

FIG. 6A is a graph showing the cycle threshold value for GAPDH for Comparative Examples D and E and Example B according to one or more aspects shown and described herein;

FIG. 6B is a graph showing the cycle threshold value fold change for GAPDH for Comparative Examples D and E and Example B according to one or more aspects shown and described herein; and

FIG. 6C is a graph showing the fold change for VEGF mRNA for Comparative Examples D and E and Example B according to one or more aspects shown and described herein.

### DETAILED DESCRIPTION

Various aspects described herein provide a method for preparation and storage of cells, tissue, or cellular material such as proteins (illustratively antibodies or other protein material), nucleic acids or other using a vitrification medium including a vitrification agent and an optional lysing agent. In various aspects, biological samples including one or more cells having nucleic acids contained therein can be contacted with the vitrification medium and vitrified to generate a storage-stable sample. In particular, various aspects enable the preservation and storage of nucleic acids at temperatures above cryogenic temperatures, and more specifically, at room temperature or above and maintain robust, well-preserved biological material. Such aspects, for example, can significantly reduce storage costs by eliminating the need for refrigeration or freezing, and can simplify the preparation of the samples for storage without adversely impacting the quality of the nucleic acids (e.g., DNA and/or RNA). Accordingly, upon reconstitution, high quality tissues, cells, proteins, nucleic acids, including RNA and DNA, may be used at a later time.

Moreover, various aspects described herein can allow saliva, blood, tissue, or other biological samples to be processed and stored with a minimal level of pre-processing, which can allow the samples to be used for any of a wide variety of purposes after storage. For example, tissue samples of tumors can be stored for extended periods of time and later processed according to the appropriate protocol, such as for use in later RNA mapping analyses. As another example, whole blood samples can be stored without first separating the plasma from the red and white blood cells, enabling any one or more of the components of the stored sample to be used at a later time.

The following terms or phrases used herein have the exemplary meanings listed below in connection with at least one aspect:

“Amorphous” or “glass” refers to a non-crystalline material in which there is no long-range order of the positions of the atoms referring to an order parameter of 0.3 or less. Transformation of a liquid into a vitreous solid occurs at the glass transition temperature T_{g}. In some aspects, the vitrification medium may be or form an amorphous material. In other aspects, the biological material may be amorphous material.

“Glass transition temperature” means the temperature above which material behaves like liquid and below which material behaves in a manner similar to that of a solid phase and enters into amorphous/glassy state. This is not a fixed point in temperature, but is instead variable dependent on the timescale of the measurement used. In some aspects, glassy state may refer to the state the biological composition enters upon dropping below its glass transition temperature. In other aspects, the glassy state may refer to the state the vitrification mixture and/or vitrification agent enters upon dropping below its glass transition temperature. In yet other aspects, the glassy state may have the mechanical rigidity of a crystal or gel, but the random disordered arrangement of molecules that characterizes a liquid.

“Crystal” means a three-dimensional atomic, ionic, or molecular structure consisting of one specific orderly geometrical array, periodically repeated and termed lattice or unit cell.

“Crystalline” means that form of a substance that is comprised of constituents arranged in an ordered structure at the atomic level, as opposed to glassy or amorphous. Solidification of a crystalline solid occurs at the crystallization temperature T_{c}.

“Vitrification” as used herein, is a process of converting a material into an amorphous material. The amorphous solid may be free of any crystalline structure.

“Vitrification mixture” as used herein, means a heterogeneous mixture of biological material(s) and a vitrification medium containing one or more vitrification agents, optionally a lysing agent, and optionally other materials.

“Biological material” or a “biological sample” as used herein, refers to materials that may be isolated or derived from a living organism(s). Examples of biological materials include, but are not limited to proteins, cells, tissues, organs, cell-based constructs, blood or fraction thereof, nucleic acids, or combinations thereof. In some aspects, biological material may refer to mammalian cells. In other aspects, biological material may refer to human mesenchymal stem cells, murine fibroblast cells, white blood cells, red blood cells, blood platelets, bacteria, viruses, mammalian cells, liposomes, enzymes, tissues (e.g. intestine, liver, neuronal, or other), or combinations thereof. In other aspects, biological material may refer to reproductive cells including sperm cells, spermatocytes, oocytes, ovum, embryos, germinal vesicles, or combinations thereof. In other aspects, biological material may refer to whole blood, red blood cells, white blood cells, platelets, blood plasma, blood serum, algae, fungi, or combinations thereof.

“Vitrification agent” as used herein, is a material that forms an amorphous structure, or that suppress the formation of crystals in other material(s), as the mixture of the vitrification agent and other material(s) cools or desiccates. The vitrification agent(s) may also provide osmotic protection or otherwise enable cell survival during dehydration. In some aspects, the vitrification agent(s) may be any water soluble solution that yields a suitable amorphous structure for storage of biological materials. In other aspects, the vitrification agent may be imbibed within a cell, tissue, or organ.

“Storable,” “storage,” or “storage-stable” as used herein, refers to a biological material's ability to be preserved and remain viable for use at a later time.

“Above cryogenic temperature,” as used herein, refers to a temperature above −80° C. Room temperature, as used herein, refers to a temperature range from greater than or equal to 18° C. to less than or equal to 37° C.

“Hydrophilic,” as used herein, means attracting or associating preferentially with water molecules. Hydrophilic materials with a special affinity for water, maximize contact with water and have smaller contact angles with water.

“Hydrophobic,” as used herein, means lacking affinity for water. Materials that are hydrophobic naturally repel water, causing droplets to form, and have small contact angles with water.

“Ambient temperature,” as used herein, refers to a temperature of from greater than or equal to about 16° C. and less than or equal to about 30° C.

Various aspects described herein provide processes for storage of biological material from biological samples. According to various aspects, a biological sample comprising one or more cells therein is contacted with a vitrification medium to form a vitrification mixture. As will be described in greater detail below, the vitrification medium comprises at least a vitrification agent and an optional lysing agent. The vitrification mixture is vitrified to generate a storage-stable sample that can be stored until further use. The storage-stable sample may later be re-hydrated and processed to extract or characterize the biological material or a portion thereof, which may then be used for quantitative, qualitative, and/or clinical analysis.

In various aspects, the vitrification medium includes at least a vitrification agent and a lysing agent. Illustrative examples of a vitrification agent include, but are not limited to, dimethylsulfoxide, glycerol, sugars (e.g. trehalose, etc.), polyalcohols, methylamines, betines, antifreeze proteins, synthetic anti-nucleating agents, polyvinyl alcohol, cyclohexanetriols, cyclohexanediols, inorganic salts, organic salts, ionic liquids, or combinations thereof. In aspects, 1, 2, 3, 4, or more vitrification agents are included in the vitrification medium.

The vitrification agent is included in the vitrification medium at a concentration that is dependent on the identity of the vitrification agent. In some aspects, the concentration of the vitrification agent below that which would be toxic to the biological sample being vitrified. As used herein, “toxic” means that functional or biological viability is not achieved upon subsequent sample use, or the biological sample is not suitable for subsequent analyses. In various aspects, the concentration of the vitrification agent is greater than or equal to 500 micromolar (μM) and less than or equal to 6 molar (M), or any value or range therebetween. As one example, trehalose is included in various aspects in a concentration of greater than or equal to 1 millimolar (mM) and less than or equal to 6 M, optionally greater than or equal to 150 mM and less than or equal to 6 M. In some aspects, the total concentration of all vitrification agents when combined is greater than or equal to 1 mM and less than or equal to 6 M, optionally greater than or equal to 1 mM and less than or equal to 6 M.

A vitrification medium as provided herein optionally includes a lysing agent. The lysing agent is included in the vitrification medium so as to partially or fully perforate or otherwise make more porous the cell membrane and optionally the nucleus to thereby facilitate transport of the vitrification agent into the cell and moisture out of the cell, thereby enabling quick vitrification. The lysing agent can be, by way of example and not limitation, a detergent. Suitable detergents for use can include sodium dodecyl sulfate (SDS), 2-[4-(2,4,4-trimethylpentan-2-yl)phenoxy]ethanol (e.g., Triton X-100), CHAPS 3-[(3-cholamidopropyl)dimethylammonio]-1-propanesulfonate), guanidine hydrochloride, other like agents, and combinations thereof. Other lysing agents are possible, provided that they do not interfere in the vitrification process or are toxic to the biological material desired. In various aspects, the lysing agent is present in the vitrification medium in an amount of from 0.01 weight percent (wt %) to 5 wt % or greater. The particular amount of lysing agent can vary depending on the particular aspect, and more specifically, the level of cell membrane permeation desired. For example, in some aspects, the lysing agent may completely lyse the cells, while in other aspects, the lysing agent may merely pierce or puncture the cell membrane to facilitate improved transfer of the vitrification agent across the membrane.

It is contemplated that, in some aspects, the vitrification medium may further include other components, such as, by way of example and not limitation, water or other solvents, a buffering agent, one or more salts, RNase or DNAse inhibitors, or combinations thereof. A buffering agent is any agent with a pKa of 6 to 8.5 at 25° C. Illustrative examples of buffering agents include choline, betaine, HEPES, TRIS, PIPES, MOPS, among others. In some aspects, the buffering agent is a buffering agent that contains large organic ions (greater than 120 kDa), such as choline, betaine, or HEPES. In aspects including a buffering agent, the buffering agent is provided at a concentration suitable to stabilize the pH of the vitrification medium to a desired level.

Salts can include, by way of example and not limitation, sodium salts, potassium salts, chloride salts, or combinations thereof. When included in the vitrification medium, the salts can be provided at a concentration of from greater than or equal to 1 millimolar (mM) to less than or equal to 500 mM. For example, the salts may be present in a concentration of from greater than or equal to 1 mM to less than or equal to 500 mM, from greater than or equal to 1 mM to less than or equal to 400 mM, from greater than or equal to 1 mM to less than or equal to 300 mM, from greater than or equal to 1 mM to less than or equal to 250 mM, from greater than or equal to 1 mM to less than or equal to 200 mM, from greater than or equal to 1 mM to less than or equal to 150 mM, from greater than or equal to 1 mM to less than or equal to 100 mM, from greater than or equal to 1 mM to less than or equal to 75 mM, from greater than or equal to 1 mM to less than or equal to 50 mM, from greater than or equal to 1 mM to less than or equal to 25 mM, from greater than or equal to 25 mM to less than or equal to 500 mM, from greater than or equal to 25 mM to less than or equal to 400 mM, from greater than or equal to 25 mM to less than or equal to 300 mM, from greater than or equal to 25 mM to less than or equal to 250 mM, from greater than or equal to 25 mM to less than or equal to 200 mM, from greater than or equal to 25 mM to less than or equal to 150 mM, from greater than or equal to 25 mM to less than or equal to 100 mM, from greater than or equal to 25 mM to less than or equal to 75 mM, from greater than or equal to 25 mM to less than or equal to 50 mM, from greater than or equal to 50 mM to less than or equal to 500 mM, from greater than or equal to 50 mM to less than or equal to 400 mM, from greater than or equal to 50 mM to less than or equal to 300 mM, from greater than or equal to 50 mM to less than or equal to 250 mM, from greater than or equal to 50 mM to less than or equal to 200 mM, from greater than or equal to 50 mM to less than or equal to 150 mM, from greater than or equal to 50 mM to less than or equal to 100 mM, from greater than or equal to 50 mM to less than or equal to 75 mM, or any and all ranges or sub-ranges included therein.

RNase and/or DNase inhibitors may be included in the vitrification medium in some aspects to prevent degradation of the nucleic acids. Any known RNase and/or DNase inhibitors known and used in the art can be used, provided they do not interfere with vitrification. However, it should be appreciated that, in various aspects, the RNase and/or DNase inhibitors may not be necessary to preserve the nucleic acids.

In some aspects, such as when the vitrification medium will be used in conjunction with a blood sample, the vitrification medium may further includes at least one anticoagulant. Alternatively, a blood sample may be collected into one or more anticoagulants and then subsequently contacted with a vitrification medium. Suitable anticoagulants can include, by way of example and not limitation, ethylene di-amine tetra acetic acid (EDTA), double oxalate, heparin, sodium citrate, sodium fluoride, and combinations thereof. When included, the anticoagulant is present in the vitrification medium or otherwise used in an amount of from 0.1 mg/mL to 5 mg/ml. In aspects, the amount of anticoagulant varies depending on the particular anticoagulant selected. For example, EDTA may be included in an amount of 1-2 mg/mL of blood, heparin may be included in an amount of 0.2 mg/mL of blood, oxalate may be included in an amount of 1-2 mg/mL of blood, and sodium fluoride may be included in an amount of 2 mg/mL of blood. As another example, sodium citrate may be included in a ratio of 1:9 where 9 parts are blood and 1 part is sodium citrate. It is contemplated that the anti-coagulant may be present in other amounts to prevent clotting of the blood sample, as will be understood by one of skill in the art.

According to various aspects, a biological sample comprising one or more biological materials therein is contacted with the vitrification medium to form a vitrification mixture. In some aspects, the vitrification mixture is incubated prior to vitrification. For example, the vitrification mixture may be incubated for greater than or equal to 5 minutes and less than or equal to 60 minutes, greater than or equal to 5 minutes and less than or equal to 45 minutes, greater than or equal to 5 minutes and less than or equal to 30 minutes, or greater than or equal to 5 minutes and less than or equal to 20 minutes. Incubation can be carried out at any suitable temperature and, in various aspects, is at room temperature (i.e., from greater than or equal to 18° C. to less than or equal to 37° C., optionally about 25° C.). Following incubation, the vitrification mixture, including the biological sample and the vitrification medium, is vitrified to generate a storage-stable sample. Vitrification can be carried out according to any known method of vitrification.

Vitrified materials are often prepared by rapidly cooling a liquid material, or small volumes of biological materials directly immersed into liquid nitrogen. The cooling reduces the mobility of the material's molecules before they can pack into a more thermodynamically favorable crystalline state. Additives that interfere with the primary constituent's ability to crystallize may produce amorphous/vitrified material. In the presence of appropriate glass forming agents, it is possible to store biological materials in a vitrified matrix above cryogenic temperatures and the vitrification can be achieved by dehydration.

Some animals and numerous plants are capable of surviving complete dehydration. This ability to survive in a dry state (anhydrobiosis) depends on several complex intracellular physiochemical and genetic mechanisms. Among these mechanisms is the intracellular accumulation of sugars (e.g., saccharides, disaccharides, oligosaccharides) that act as a protectant during desiccation, trehalose is one example of a disaccharide naturally produced in desiccation tolerant organisms.

Sugars like trehalose may offer protection to desiccation tolerant organisms in several different ways. A trehalose molecule may effectively replace a hydrogen-bounded water molecule from the surface of a folded protein without changing its conformational geometry and folding due to the unique placement of the hydroxyl groups on a trehalose molecule. A sugar molecule may also prevent cytoplasmic leakage during rehydration by binding with the phospholipid heads of the lipid bilayer. Furthermore, many sugars have a high glass transition temperature, allowing them to form an above cryogenic temperature or a room temperature glass at low water content. The highly viscous ‘glassy’ state reduces the molecular mobility, which in turn prevents degradative biochemical reactions that lead to deterioration of cell function and death as well as the degradation of proteins and nucleic acids.

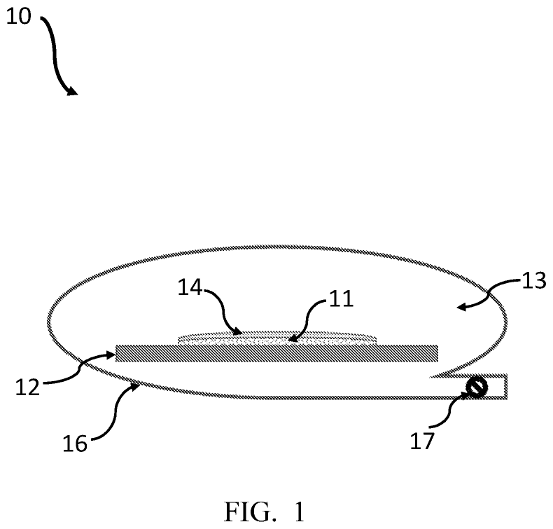

In some aspects, the vitrification of the biological sample comprises dehydration in the presence of glass forming sugar trehalose, such as has been disclosed in N Chakraborty, et al., Biopreservation and Biobanking, 2010, 8 (2), 107-114. With reference to FIG. 1, the system 10 is the most common approach to dehydrating a biological material. A sessile droplet 11 is placed on a substrate 12 and evaporatively desiccated in an enclosure 16 having low humidity environment 13. The humidity, pressure and temperature inside the enclosure can be operably controlled by a control device 17. However, desiccation using evaporative drying of sessile droplets of the system 10 is inherently slow and non-uniform in nature. A glassy skin forms at the liquid/vapor interface 14 of the sample when the biological materials are desiccated in glass forming medium. This glassy skin slows and ultimately prevents further desiccation of the sample beyond a certain level of dryness and induces significant spatial non-uniformity of the water content across the sample. As a result, cells trapped in the partially desiccated sample underneath the glassy skin may not vitrify but degrade due to high molecular mobility.

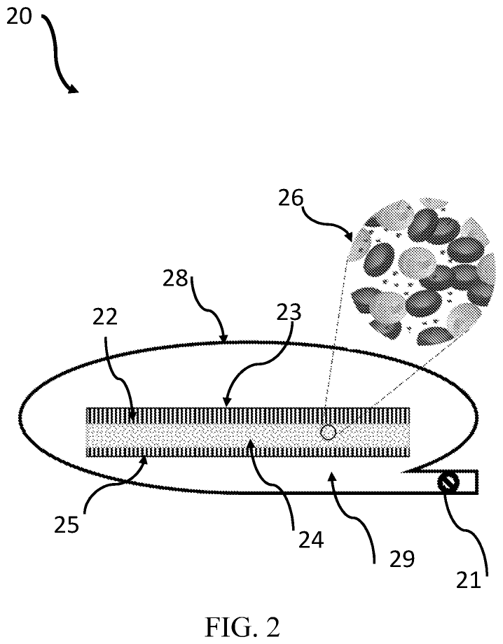

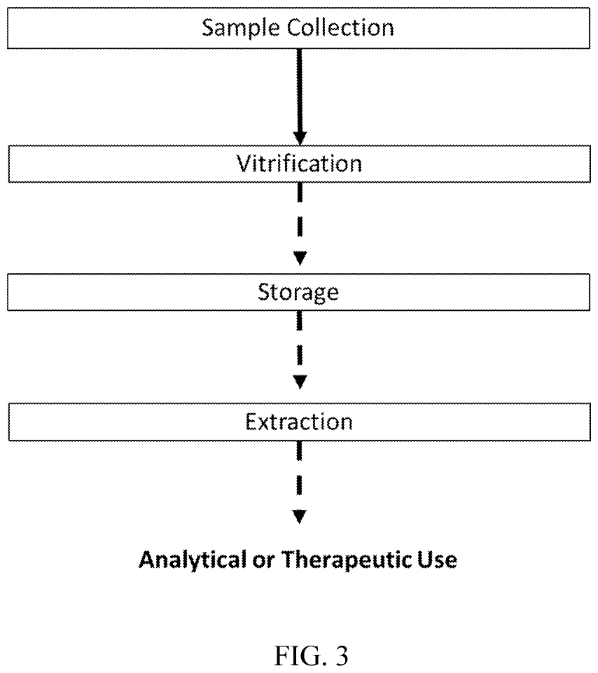

In other aspects, the vitrification of the biological sample comprises dehydration in the presence of glass forming sugar trehalose using capillary assisted drying, such as has been disclosed in U.S. Pat. No. 10,433,540. An example device for performing such processes is shown in FIG. 2.

A process of vitrification may be performed by vitrification on or within a membrane that may include within the membrane one or more capillary channels, optionally continuous capillary channels. Capillaries can provide an interface for rapid evaporation. A membrane may be formed of a plurality of capillary channels, optionally a plurality of contiguous capillary channels. The capillary network formed from a porous material such as a membrane may be made of a material that is not toxic and not reactive to the biomaterials or biological samples and does not react chemically or physically with the vitrification medium. A membrane may be made of a material that is hydrophilic or has been modified to be hydrophilic. In some aspects, optionally using a support structure as discussed below, a membrane may be partially soluble or have time/stimulus dependent solubility in either the vitrification mixture or the reconstitution solution. The membrane material can be of a suitable polymer, metal, ceramic, glass, or a combination thereof. In some aspects, a contiguous capillary network is formed from a material of polydimethylsiloxane (PDMS), polycarbonate, polyurethane, polyethersulphone (PES), polyester (e.g. polyethylene terephthalate), among others. Illustrative examples of a capillary channel containing membrane suitable as a surface in the devices and processes provided herein include hydrophilic filtration membranes such as those sold by EMD Millipore, Bellerica, Mass. In certain aspects, the porous material does not substantially bind, alter, or otherwise produce a chemical or physical association with a component of a biological sample and/or vitrification medium. The porous material is optionally not derivitized. Optionally, capillary channels may be formed in a substrate (e.g. desiccation chamber walls) of desired material and thickness by PDMS formation techniques, laser drilling, or other bore forming technique as is known in the art.

In some aspects, the capillary network provided by a porous material includes pores with a cross sectional dimension of about 100 μm or less, optionally 20 μm or less, such that the pores provide underlying capillaries to assist in vitrification. When a capillary network is being used to vitrify a tissue, a larger pore size such as a cross sectional dimension of about 100 μm or less may optionally be used. When a cellular sample (not tissue) is used in some aspects, a cross sectional dimension of about 20 μm or less may be used. In some aspects, the pores may be of an average opening of from about 100 μm to about 0.1 μm, including about 90, 80, 70, 60, 50, 40, 30, 20, 19, 18, 17, 16, 15, 14, 13, 12, 11, 10, 9, 8, 7, 6, 5, 4, 3, 2, 1.0, 0.9, 0.8, 0.7, 0.6, 0.5, 0.4, 0.3, and 0.2 μm. A capillary channel may have a length optionally defined by the thickness of a substrate that forms the channels or by one or a plurality of individual channels themselves. A capillary channel length is optionally about one millimeter or less, but is not to be interpreted as limited to such dimensions. Optionally, a capillary channel length is of about 0.1 microns to about 1000 microns, or any value or range therebetween. Optionally, a capillary channel length is of about 5 to about 100 microns, optionally of about 1 to about 200 microns, and/or optionally of about 1 to about 100 microns. A capillary channel length is optionally about 5, 10, 15, 20, 25, 30, 35, 40, 45, 50, 55, 60, 65, 70, 75, 80, 85, 90, 95, or 100 microns. In some aspects, the length of the capillary channels varies throughout a plurality of capillary channels, optionally in a non-uniform variation.

The cross-sectional area of the capillary channel(s) may be of about 8000 μm² or less, optionally 2000 μm² or less. Optionally a cross-sectional area is of about 0.01 μm² to about 8000 μm², optionally of about 100 μm² to about 2000 μm², or any value or range therebetween. Optionally, a cross-sectional area of the capillary channel(s) is of about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000 μm² or less.

As shown in FIG. 2, a capillary assisted vitrification device 20 includes a capillary plate/membrane 22 positioned within an enclosure 28. A biological sample is illustrated within the pores of the membrane. Optionally, in some aspects a capillary membrane may be replaced by several filter layers or other membrane that does not have a plurality of well-defined channels. In other aspects, a plurality of membranes may be used with a biological sample sandwiched between the membranes or otherwise housed within a plurality of membranes that when layered together form a vitrification membrane suitable for vitrification of the biological sample. The plate/membrane 22 optionally includes a plurality of capillary channels, optionally substantially parallel capillary channels, each having a first opening 23 and a second opening 25. The vitrification mixture 24 is placed on the first opening 23, which finds its way into the capillary channels, further exposing the vitrification mixture's 24 surface to a surrounding atmosphere 29 through the second openings 25. In various aspects, the surrounding atmosphere 29 has a humidity that is lower than the humidity of the vitrification mixture. Vitrification is accomplished by desiccating away the vitrification mixture by capillary action until the vitrification mixture enters into a glassy state. The chemistry, humidity, pressure and temperature inside the enclosure 28 are controlled through one or more control mechanisms 21.

The control mechanism 21 is simplified for illustration purposes only and can have multiple systems and mechanisms to attain the most favorable conditions for desiccation and vitrification. In some aspects, the vitrification mixture 24 is optionally sandwiched between two plate/membranes similar to 22 to benefit from the capillary assisted drying method at the top and bottom surfaces of vitrification mixture 24.

In some aspects, a flow of low humidity gas (less than 30% relative humidity) is provided across the second openings 25 of the capillary plate/membrane, or the opposite side of the membrane as the vitrification medium so as to enhance the capillary effect. Inert or relatively inert gases such as nitrogen, argon, xenon, or others may be used as a low humidity gas. In some aspects, a reduced pressure or vacuum is maintained inside the enclosure 28. In some aspects, a suction force/pressure is provided across the second opening 25 to achieve increased desiccation speed. It is to be noted that, maintaining a low humidity surrounding (optionally 5% relative humidity or less) is essential to prevent rehydration after desiccation has been performed. Additional details on capillary assisted drying may be found in U.S. Pat. No. 10,433,540.

The vitrification method may be performed at a temperature from −80° C. to +60° C. The temperature range is optionally where the mobility of water molecules in the sample is high and the temperature is not detrimental to the health and viability of the biological material. This would vary from material to material as well as the composition of the vitrification medium. In some aspects, the vitrification temperature is 0.1° C. to 40° C. Optionally, the vitrification temperature is 4° C. to 26° C. Optionally, the vitrification temperature is about 25° C.

The vitrification method may be performed in a dry atmosphere or environment. A dry environment is an environment with a humidity level below saturation. In some aspects, the humidity level of the environment, such as the environment on the second side of the capillary tube is 30% relative humidity or less, optionally 20% or less, optionally 10% or less, optionally 5% or less. A dry environment optionally has a humidity between 1% and 30% or any value or range therebetween, optionally between 1% and 5%.

The vitrification method may be performed in a low pressure environment (less than 1 atm (760 mmHg)). A low pressure environment will have favorable impact on the rate of vitrification. The environmental pressure is optionally 100 mmHg or 0.1 atm. Optionally, the environmental pressure is from 10 mmHg to 760 mmHg, or any value or range therebetween. Optionally, the environmental pressure is from 10 mmHg to 200 mmHg.

The vitrification method may be performed for a desiccation time. A desiccation time is a time sufficient to promote suitable drying to vitrify the vitrification medium. A desiccation time is optionally 1 second to 1 hour. Optionally, a desiccation time is 1 second to 50 min, optionally 5 seconds to 60 min. Desiccation time may vary dependent on the sample type or physical characteristics and the particulars of the capillary channels.

In still other aspects, vitrification can be carried out on or between membranes or filter paper(s). Other methods of vitrification can be used depending on the particular aspect.

After vitrification, the sample is storage stable, and can be stored at temperatures above cryogenic temperatures while remaining viable and not substantially degrading for use at a later time. In some aspects, following vitrification, the vitrification mixture may be enclosed in a glass state in a protective enclosure that is impervious to water and air and stored. In some aspects, the storage-stable sample can be stored at a temperature of from greater than or equal to −196° C. to less than or equal to +60° C. or greater, from greater than or equal to 16° C. to less than or equal to 60° C. or greater, or from greater than or equal to 18° C. to less than or equal to 60° C. or greater for a period of time before it is used. In some aspects, the storage time is greater than or equal to 1 day, greater than or equal to 5 days, greater than or equal to 10 days, greater than or equal to 20 days, greater than or equal to 30 days, greater than or equal to 45 days, greater than or equal to 60 days, or more.

In various aspects, when the storage-stable sample is ready for use, it is rehydrated (or reconstituted) and processed according to the particular protocol in which the sample is being used. In some aspects, the storage-stable sample can be rehydrated using a rehydration solution that is used to precipitate proteins or one or more types of nucleic acid. In aspects, the storage-stable sample is reconstituted using a lysis buffer, such as may be included to fully lyse cellular material in an extraction and/or purification kit to be used to process the storage-stable sample.

Although the sample may be used in a variety of protocols, in some aspects, the sample is processed to extract nucleic acids, such as DNA and/or RNA. For example, following rehydration, the DNA and/or RNA may be pelleted, bound, washed, eluted, dried and/or dissolved depending on the particular extraction method employed.

In some aspects, the RNA is extracted according to the GITC-based method. Accordingly, the storage-stable sample is rehydrated, allowed to phase separate, and propanol is added to the supernatant. The mixture is then centrifuged to form a pellet comprising the RNA. Next, the RNA pellet is washed, dried, and dissolved for analysis.

In some aspects, the RNA is extracted according to the TRIspin method. Accordingly, the storage-stable sample is rehydrated, allowed to phase separate, and ethanol is added to the supernatant. The RNA is bound, washed, and eluted, and may then be used for analysis.

In some aspects, the RNA is extracted using a column-based method. In this aspect, the storage-stable sample is rehydrated, and ethanol is added. The RNA is bound, washed, and eluted, and may then be used for analysis.

Other methods for extraction are contemplated. Accordingly, regardless of the particular method of extraction employed, in some aspects, the cell lysis step which is typically the first step of any such extraction methods, can be performed during the vitrification process, as described hereinabove, and the remaining steps can be performed following rehydration. Thus, aspects described herein can enable faster vitrification while providing storage-stable samples that can be used in any one of a variety of processes following storage.

In some aspects, DNA or RNA may be extracted from the vitrified cells. For example, any one of a number of commercially available DNA or RNA extraction kits or similar processes can be used to extract DNA or RNA from the storage-stable sample. In various aspects, the lysis buffer used in the extraction kit may be used to reconstitute the sample.

Moreover, various aspects described herein can enable whole blood or a portion thereof to be preserved and stored above cryogenic temperatures. For example, whole blood, serum, plasma, red blood cells, platelets, and/or lymphocytes can be collected into a tube (optionally with an anticoagulant) or on a membrane, contacted with the vitrification medium, incubated for 5-20 minutes at ambient temperature, and vitrified. The sample can be stored at ambient temperature until use. Extraction of DNA, RNA, and/or proteins may be carried out by processing the sample with trizol. Accordingly, storage costs can be reduced, and the preserved blood samples can be used for any one of a variety of processes following storage, providing greater flexibility for the samples. For example, the storage-stable sample can be rehydrated and the leukocyte-lysate can be extracted in lysis buffer. The lysate may be transferred with ethanol to a spin column and washed with wash buffer. The total RNA may then be eluted with RNase-free water, and the RNA can be used for quantitative, qualitative, and clinical analysis.

In some aspects, whole blood may be collected, centrifuged, and one or more of the layers (e.g., the plasma layer, the buffy coat, or the erythrocyte layer) may be transferred into a matrix of the vitrification medium and subjected to vitrification. After storage, the sample may be reconstituted with a diluent (such as PBS liquid, with or without protease inhibitors) and subjected to qualitative and/or quantitative analysis.

Although various aspects have been described herein with regard to the use of a biological sample in the form of whole blood, it is further contemplated that other types of biological samples may be used, as generally described herein. In some aspects, the biological sample may be in the form of a tissue. In some such aspects, the tissue may be homogenized using a lysis solution, pulverized, or subjected to enzymatic digestion. In alternative aspect, a tissue may be subjected to cryosection methods that may be used according to various aspects.

In some aspects, a biological sample is heterogeneous. A heterogeneous sample is a sample that contains one or more foreign (non-subject derived) organisms that also include nucleic acid that may be simultaneously stored with the biological sample and may contaminate the results of downstream analyses of the sample. As such, an additional preparation step may be desired to selectively isolate the desired biological sample components relative to the undesired contaminants. As a non-limiting example, a biological sample may be a saliva. Saliva is known to include cells from both a host organism as well as contaminating bacteria or viruses.

A heterogeneous sample may be subjected to a pretreatment step to remove or reduce the amount of foreign organism (optionally bacterial, virus, yeast, or other) in the sample. A pretreatment step may be combined with a vitrification step, but in some aspects, a pretreatment step occurs prior to actual vitrification. A pretreatment step may be contacting a biological sample with an isolation medium, optionally an particulate or membrane form, that contains or is bound to a molecule selective for one or more foreign organisms, optionally bacterial, viral, yeast, and/or other non-subject organism. In some aspects, a sample is placed in contact with a surface of an isolation media that includes an isolation agent specific for one or more non-subject organisms, optionally a mannose-binding lectin (MBL), optionally with the sequence of NCBI Reference Sequence: NP_000233. MBL is a C-type lectin that binds N-acetylglucosamine residues and mannose on bacteria, yeast and some parasites and viruses. By contacting a biological sample with a surface that includes MBL, the non-subject organism may be selectively sequestered from subject cells thereby improving the ability of the process to selectively isolate and optionally vitrify subject cells in a biological sample.

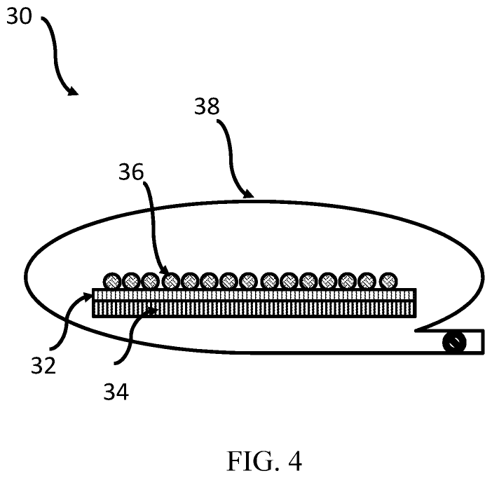

Now referring to FIG. 4, in some aspects, a sample is subjected to a membrane system whereby at least one isolation membrane 32 is used to remove foreign organisms. FIG. 4 illustrates a plurality of particles 36 that includes an isolation agent bound to the surface thereof. The particles may be incubated with a vitrification mixture and/or a biological sample. The particles selectively bind to a foreign organism with one more isolation agents, optionally a mannose-binding lectin (MBL), bound thereto. The sample with the particles is placed on an isolation membrane 32 with a sufficient pore size to allow passage of a biological sample, while retaining the particles on the surface thereof.

Alternatively, or in addition, an isolation membrane itself includes one or more isolation agents bound thereto whereby a biological sample or vitrification mixture is contacted to the isolation membrane whereby the foreign organisms are bound to the isolation membrane while the desired material in the biological sample pass through the isolation membrane to contact a vitrification membrane for subsequent vitrification of the biological sample. An isolation membrane may be any membrane system that is substantially porous such that cellular material may pass through the membrane absent being bound by an isolation agent therein or thereon.

The isolation media can be of a suitable polymer (e.g. polyvinylidene difluoride (PVDF) or the like), metal, ceramic, glass, or a combination thereof. In some aspects, an isolation media may be made from PVDF, cellulose ester, nitrocellulose, or other desired material. One or more isolation agents may be bound to or otherwise associated with a suitable isolation media. When a biological sample is then contacted with the isolation media, foreign cells/organisms may be selectively bound to the isolation agent whereby subject cells may pass through the system for capture or collection and subsequent vitrification as otherwise provided herein.

An isolation membrane, optionally with an isolation agent, may be layered upon a vitrification membrane suitable for vitrification of a biological sample as otherwise provided herein. A vitrification membrane may be any material that includes or defines a capillary network. Optionally, such a porous material membrane may be made of a material that is not toxic and not reactive to the biomaterials or biological samples and does not react chemically or physically with the vitrification medium. The material can be of a suitable polymer, metal, ceramic, glass, or a combination thereof. In some aspects, a contiguous capillary network is formed from a material of polydimethylsiloxane (PDMS), polycarbonate, polyurethane, polyethersulphone (PES), polyester (e.g. polyethylene terephthalate), among others. Illustrative examples of a capillary channel containing membrane suitable as a surface in the devices and processes provided herein include hydrophilic filtration membranes such as those sold by EMD Millipore, Bellerica, Mass. In certain aspects, the porous material does not substantially bind, alter, or otherwise produce a chemical or physical association with a component of a biological sample and/or vitrification medium. The porous material is optionally not derivitized. Optionally, capillary channels may be formed in a substrate (e.g. desiccation chamber walls) of desired material and thickness by PDMS formation techniques, laser drilling, or other bore forming technique as is known in the art.

After a biological sample has passed through an isolation membrane with an isolation agent to isolate or remove non-subject cellular material, the remaining cellular material is collected within or on the membrane for subsequent vitrification and optional storage. An isolation membrane may be layered on top of a vitrification membrane. Following contact with the biological sample, the isolation membrane may be removed for analysis or discarded and the remaining biological sample vitrified on or within the vitrification membrane by the processes as described herein. Alternatively, the isolation membrane may remain associated with the vitrification membrane and the entire membrane system subjected to vitrification as provided herein. The isolation membrane may then be peeled away or otherwise removed from the vitrification membrane prior to reconstitution of the biological material therein. The result of the pretreatment step is a substantial removal of non-subject cellular material thereby improving the isolation and storage of subject biological sample material.

Optionally, the isolation agent may be bound to beads or particles. In some aspects, the beads with bound isolation agent may be mixed with the biological sample and vitrification mixture prior to pouring the vitrification mixture onto the membrane system. It will be appreciated that such a step will provide sufficient contact time between the beads and the vitrification mixture to capture the desired contaminating pathogens. Now referring to FIG. 4, upon pouring the vitrification mixture onto the membrane system, beads with captured pathogens will be isolated from the vitrification mixture and remain on the top surface of the membrane system. For this to materialize, the pore size of membrane system should be smaller than the bead size and larger than the size of the cells that must pass into the vitrification membrane. The size of the beads can be between 6 microns and 500 microns, optionally 10 microns and 500 microns. The beads can made of a polymer such a polystyrene or iron oxides, such as magnetite (Fe₃O₄) functionalized to bind to the isolation agents.

In various aspects as also provided herein a biological sample may be a tissue or other portion of an organism. Storing tissue samples in non-cryogenic conditions is typically difficult. Simple vitrification of tissue samples does not lead to adequate stability of the tissue material due to uneven drying or lack of maintenance of tissue structure. As such provided are methods for vitrifying tissues that not only preserve the molecular materials of the tissue, but also maintain the structure and other characteristics of the overall tissue itself thereby dramatically improving the validity and ability to perform subsequent tissue analyses. A tissue that may be used as a biological sample in the processes as provided herein include, but are not limited to tissues from sources that are neuronal, liver, heart, kidney, blood vessel, kidney, lung, larynx, stomach, esophagus, pancreas, thyroid, muscle, epithelial, hair, or any other recognized biological tissue type.

In a vitrification process as provided herein, a tissue sample may be penetrated with a vitrification agent (optionally a sugar) and contacted with a polymer or polymer forming agent (e.g. PEG hydrogel). For example, the tissue may be injected with a trehalose-PEG hydrogel precursor and an attachment agent suitable to covalently, ionically, or otherwise associate a tissue or portion thereof with a polymer.

A polymer may be any molecule that may be used as or to create a polymer suitable for storing a tissue above cryogenic temperature includes but are not limited to: polyalkyl alcohols and glycols such as polyoxyethyelene or polyoxyethylene derivatives; neophenyl glycol diacrylate (NPGDA), polyethylene oxide (PEO), polyacrylamide (PAAm), polyhydroxyethyl methacrylate (PHEMA), polyacrylic acid (PAA), polyvinyl alcohol (PVA), poly (N-isopropylacrylamide) (PNIPAM), polyvinylpyrrolidone (PVP), polylactic acid (PLA), polyglycolic acid (PGA), polycaprolactone (PCL), gelatin, alginate, carrageenan, chitosan, hydroxyalkylcellulose, alkylcellulose, silicone, rubber, agar, carboxyvinyl copolymer, polydioxolane, polyacrylic acetate, polyvinylchloride, maleic anhydride, styrene and styrene polymers; dextrans; heparin and polymers of heparin; polypeptides of glutamate, aspartate, or combinations thereof.

A polymer is optionally linear, branched, liable, or combinations thereof. The polymers are optionally homomeric or heteromeric. Illustrative examples of polymeric moieties include one or more molecules of carbohydrate or polyoxyethylene (otherwise known as polyethylene glycol or “PEG”).

A polymer is optionally a polyethylene glycol. A polyethylene glycol optionally includes from 2 to 20000 ethylene glycol units. Optionally, the number of ethylene glycol units is from 2 to 10000, optionally from 2 to 5000, optionally from 2 to 2000. In various aspects, a polyethylene glycol (PEG) is a derivative of a polyethylene glycol including but are not limited to polyethylene glycol-vinylsulfone. The PEG may be a linear or branched PEG molecule. Optionally, a branched PEG may be a 2, 4, 6, 8, or other arm PEG molecule.

In some aspects, a process further includes adding a crosslinking agent with the polymer forming agent. A crosslinking agent is any agent suitable for linking two or more monomers/polymers together. A crosslinking agent optionally has one or more acrylate or methacrylate functional groups. Illustrative crosslinking agents include, but are not limited to 2-hydroxyethyl methacrylate (HEMA), acrylic acid, and methacrylic acid, adipic acid hydrazide diamide acrylate, acrylamide, methacrylamide, alkyl-(meth) acrylamide. N-mono (meth) acrylamide, N,N-di-C1-C4 alkyl-(meth) acrylamide (N,N-di-C1-C4 alkyl-(meth) acrylamide), N-butyl (meth) acrylate, N-butyl (meth) acrylate, methyl (meth) acrylate, ethyl (meth) acrylate, kids isobornyl (meth) acrylate, cyclohexyl (meth) acrylate, hydroxyethyl acrylate, hydroxyethyl methacrylate, hydroxypropyl acrylate, hydroxypropyl methacrylate, hydroxybutyl acrylate, N-(2-hydroxyethyl) acrylamide [N-(2-hydroxyethyl) acrylamide], N-methyl acryl-amide, N-butoxymethyl acrylamide, N-methoxymethyl acrylamide, N-methoxymethyl methacrylamide, 2-acrylamidoglycolic acid and 2-carboxyethyl 2-carboxyethyl acrylate, 2-hydroxy-5-methoxyacetophenone, 2-hydroxyethyl cellulose and 2-hydroxyethyl disulfide, and the like, or combinations thereof.

A process optionally further includes one or more attachment agents present within a vitrification mixture. Optionally, an attachment agent is boronic acid. Without being limited to one particular theory, it is believed that the boronic acid may covalently bond the polymer to membrane proteins of the cells of the tissue. Accordingly, a hydrogel forms between the cells of the tissue and provides support to the cells and tissue to prevent shrinkage or collapse during vitrification and sectioning. After the hydrogel is formed, the tissue can be vitrified. The vitrified samples may then be effectively sectioned, if desired. Once the tissue has been sectioned, a mixture of glucose and water can be added to decouple the boronic acid, enabling the hydrogel to decouple from the membrane proteins. The boronic acid and hydrogel can then be washed away, leaving the tissue sample, including its original architecture as preserved by the vitrification process.

Such aspects can enable histologic analysis of samples. Conventional tissue processing for histology includes the infusion of a low melting point paraffin or agarose into the tissue prior to freezing or vitrifying the sample prior to sectioning the tissue sample. Although paraffin provides rigidity needed to maintain the cell architecture, it results in tissue contamination. Agarose impacts RNA extraction, and sticks to the tissue wall. Accordingly, the use of a trehalose-PEG hydrogel (as one example provided herein) can enable vitrification of the tissue while providing support for the cell architecture without contaminating or adversely impacting downstream processing, since the hydrogel can be debound and washed away through a glucose-water wash step. Other advantages will be apparent to those skilled in the art.

Optionally, a vitrification medium includes a switchable support material with the capability to switch between relatively high and low viscus states triggered by two different stimuli. Before applying stimuli, a switchable material will behave as a low-viscous liquid with excellent flowability, allowing use for in situ perfusion into the tissue matrix by processes as provided herein. Once the first stimulus is applied, the material will switch from low-viscous liquid (sol state) to yield-stress fluid (gel state), providing sufficient rigidity and support to substantially maintain tissue and/or membrane structural configuration during vitrification. When the second stimulus is applied, the support material will switch from rigid material back to low-viscous liquid, allowing easy removal.

In some aspects, light of varying wavelengths may be used as stimuli. Optionally, ultraviolet (UV) and visible light will be used as the switching stimuli. In some aspects, a photo-switchable hydrogel support will exhibit rigidity under visible light, but will be able to completely dissociate into solution state under UV radiation. An exemplary photo-switchable material is a supramolecular hydrogel formed by azobenzene (azo) and cyclodextrin (CD) host-guest complexes, optionally as described by Vapaavuori, et al., J. Mat. Chem. C., 2018; 6:2168-2188 or Rosales, et al., Bioconjugate Chem., 2018; 29: 905-913. There are three main types of cyclodextrins with different cavity sizes: α-CD, β-CD, and γ-CD. They include six, seven, and eight linked glucopyranose sub-units, respectively, forming a cone structure. The hydroxyl groups are located outside of the cone, making it hydrophilic. However, the inside of the cone is hydrophobic, so it can host hydrophobic molecules such as azobenzene in aqueous solution, forming host-guest complexes. Azobenzene (azo) is a well-known photo-responsive molecule. Under visible light (˜520 nm), it is in a thermodynamically stable trans-state. When irradiated with UV (˜375 nm), it photo-isomerizes into the cis-isomer. Trans-azo has a geometry that allows it to enter the cavities of CD driven by hydrophobic interaction in an aqueous solution, forming a host-guest complex. When it is photo-isomerized into the cis-azo, its geometry no longer fits into the CD cone, leading to dissociation of the complex. Using the photoreversible azo-CD complex as the crosslinker, photo-switchable hydrogels can be prepared that gel under visible light but readily dissociate into liquids (sol) under UV radiation. This sol-gel switching is reversible and can be completed within two minutes. Other methods and materials for forming a switchable support material can be found in Koopmans and Ritter, Macromolcules, 2008; 41:7418-7422.

A process as provided herein thereby optionally further includes a switchable support material within a vitrification medium and applying a stimulus to covert the switchable support material to a viscous state optionally followed by subjecting the vitrification medium to a vacuum vitrification process as provided herein to thereby store a tissue or other cellular material in a vitrified state with sufficient support to maintain other physical and/or chemical characteristics of the biological sample.

### EXAMPLES

The following examples are provided to illustrate various aspects, but are not intended to limit the scope of the claims. Approximate properties, characters, parameters, etc. are provided below with respect to various work examples, comparative examples, and the materials used in the working and comparative examples.

### Example 1

Cell Culture: LINTERNA Jurkat T-cells (stably expressing tGFP with G418 resistance gene) were obtained from Innoprot, Spain. Cells were cultured in cultured in at 37° C. and 5% CO₂ in RPMI 1640 (Gibco), 10% heat inactivated fetal bovine serum (Hyclone), 1× Glutamax (Gibco) and G418 (Gibco). Cultures were maintained in 25-cm² T-flasks (Corning Incorporated, NY) at 37° C. and equilibrated with 10% CO₂-90% air. A fresh culture medium was replaced every three days to maintain the cells.

For Example A, a sample of 5×10⁶ Jurkat T-cells were incubated for 10-15 min. in 250 μl vitrification media containing 600 mM Trehalose, 5% Glycerol, 0.01% Triton X-100. Then the samples were sandwiched between two PES membrane scaffolds with a 1.2 micrometer pore size and vitrified in a vacuum of −29 mmHg for about 6 min to achieve a moisture residual ratio (MRR) of 0.01. Then the samples were stored at 25° C. or 55° C. for 3 days prior to RNA extraction.

RNA was extracted using a PureLink RNA Mini Kit (Invitrogen). The vitrified cells were rehydrated with 0.6 ml of lysis buffer containing 1% 2-mercaptoethanol and incubated for 15 min at room temperature. The lysate was homogenized by passing the lysate 10 times through a 21-gauge syringe needle. An equal volume of 70% ethonal was added to the lysate. Then, the lysate/supernatent were extracted by centrifugation (12,000×g for 15 secs at room temperature) and passed through the RNA binding spin column by centrifugation at 12,000×g for 15 secs at room temperature. Next, DNA contamination was removed by DNase digestion on the column (PureLink DNase Set, Invitrogen) followed by two step washing (centrifugation at 12,000×g for 15 seconds) with 700 μl of Wash Buffer-I and 500 μl of Wash Buffer-II with ethanol to remove the contaminant and inhibitors. The spin column with bound RNA was dried for 2 mins and 50 μl of RNase-free water was added to the spin column and incubated for 2 minutes. The pure RNA was eluted by centrifugation at 12,000×g at room temperature in a new tube.

As a control, RNA was extracted from a sample of the identical number of fresh cells using the same techniques as the vitrified cells (Comparative Example A).

For Comparative Example B, a sample containing the same number of cells was cryogenically frozen and stored at −80° C. for 2 hours. The cells were thawed and RNA extracted according to the same RNA extraction process as was used for Example A.

For Comparative Example C, 5×10⁶ Jurkat T-cells were incubated for 10-15 min in 250 μL vitrification media containing 600 mM Trehalose and 5% Glycerol, the vitrification media excluding Triton X-100. The sample was vitrified for about 6 min to achieve an MRR of 0.01. Then the sample was stored at room temperature for 3 days. The cells were then rehydrated in lysis buffer and RNA was extracted using the same techniques as the vitrified cells.

The same vitrification protocols for Example A and Comparative Example C were performed and cells were stored at either 25° C. or 55° C. for three days followed by RNA extraction.

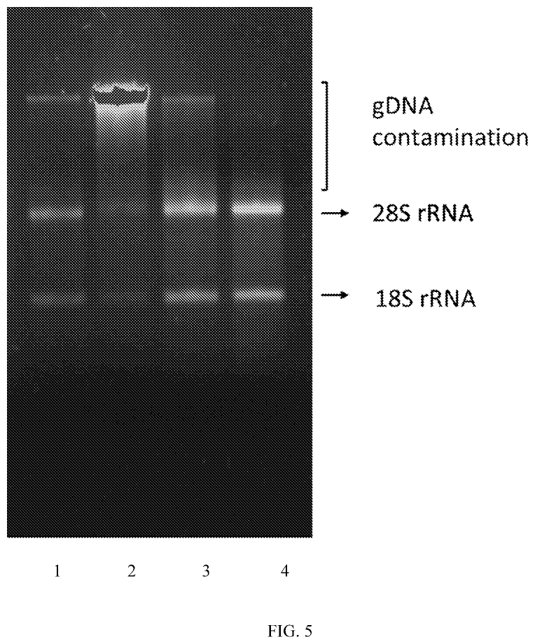

For all samples, 5 μg of RNA was loaded per well on a 1.2% agarose gel (Native Sybr-safe) and electrophoresed to enable separation. An image of the gel is provided as FIG. 5. Intact total RNA run on a denaturing gel will have sharp 28S and 18S rRNA bands (eukaryotic samples). The 28S rRNA band should be approximately twice as intense as the 18S rRNA band. This 2:1 ratio (28S:18S) is a good indication that the RNA is intact. Partially degraded RNA will have a smeared appearance, will lack the sharp rRNA bands, or will not exhibit a 2:1 ratio, as can be seen for Comp. Ex. A (lane 1) and Comp. Ex. C (lane 3). Completely degraded RNA will appear as a very low molecular weight smear (Comp. Ex. B; lane 2). As can be seen in FIG. 5A, Example A stored at 55° C. for 1 week (lane 4), which was vitrified and stored according to various aspects described herein, have sharp 28S and 18S rRNA bands with a good intensity ratio, indicating that the RNA is intact following vitrification and storage at room temperature.

Similar results are obtained by RNA quantification. RNA prepared as above for each sample was quantified spectrophotometrically with results illustrated in Table 1.

TABLE 1

| Sample | Name | 260/280 | ng/μL |
|---|---|---|---|
| Comp. Ex. A | Fresh Jurkat Cells | 2.07 | 378.353 |
| Comp. Ex. B | Jurkat Cells[1-2 h@−80° C.] | 1.889 | 22.833 |
| Comp. Ex. C | Cells + VM Vitrified | 2.111 | 122.273 |
| Ex. A | Cells + VM + TRITON Vitrified | 2.113 | 202.033 |

Vitrification in the presence of the lysing agent Triton-X100 showed a clear improvement in intact RNA relative to cells vitrified in the absence of the lysing agent. Comparative Example B was almost totally degraded. The improved RNA quality was observed when the vitrification media included the lysing agent independent of storage at 25° C. or elevated temperature of 55° C. for 3 days illustrating the robust capability to store cellular samples prepared as described herein.

### Example 2

LINTERNA Jurkat T-cells were obtained and cultured as in Example 1.

A sample 5×10⁶ Jurkat T-cells were incubated for 10-15 min. in the 250 μl vitrification media containing 600 mM Trehalose, 5% Glycerol, 0.01% Triton X-100. Then the samples were vitrified for about 6 min to achieve an MRR of 0.01. Then the samples were stored at either 25° C. or 55° C. for 3 days prior to RNA extraction. RNA extraction was performed as in Example 1.

As a comparator, RNA was extracted from a sample of the fresh cells stored at 4° C. prior to RNA extraction and analysis as per the vitrified cells.

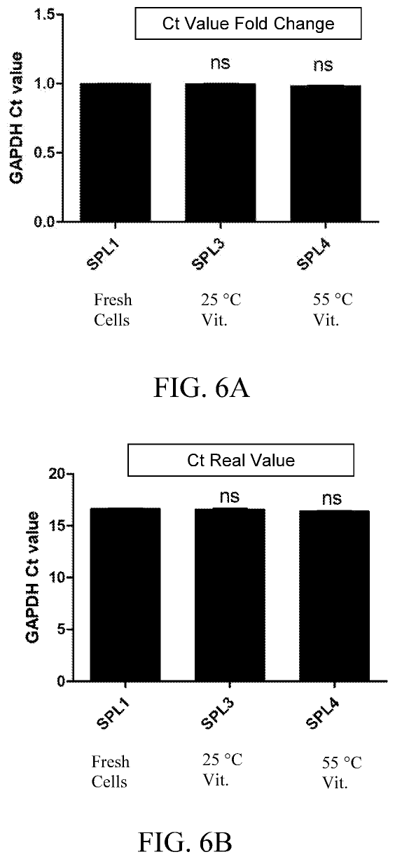

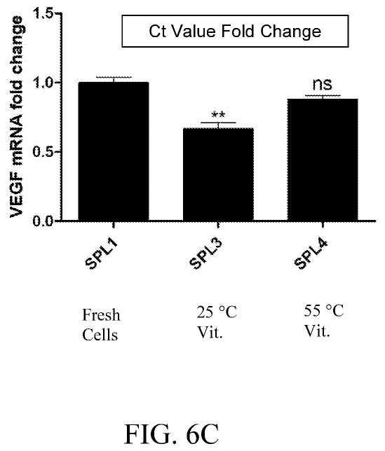

RNA extracted from each of the samples was then subjected to RT-PCR using an RNA template for VEGF (member of the PDGF/VEGF growth factor family) or GAPDH. FIG. 6A shows the cycle threshold (Ct) values of GADPH mRNA from cells from the various preparation and storage techniques. FIG. 6B shows the Ct value fold change values of GADPH mRNA from cells from the various preparation and storage techniques. FIG. 6C shows the VEGF mRNA fold change from cells from the various preparation and storage techniques. As shown in FIGS. 6A and 6B, there was no significant change between the Ct values for the fresh cells and the vitrified cells at both storage temperatures for the GAPDH gene. In addition, robust VEGF mRNA was observed (FIG. 6C). Accordingly, it was concluded that the vitrification processes described herein could be used to vitrify and store cells without degrading the VEGF mRNA.

### Example 3

Various animal tissues were obtained and subjected to vitrification. Samples of mouse intestine and liver were humanely collected by an approved animal protocol. Tissues were cut into small sections (about 1 mm thickness, weight 15 mg) and incubated for 20 min in vitrification media including 600 mM Trehalose, 5 wt % Glycerol, 0.5 wt % Triton X-100. The samples were vitrified as per Example 1 for 5 min to achieve an MRR of 0.01 and stored at either 25° C. or 55° C. for one week.

A second set of samples is vitrified in the same vitrification medium further including a trehalose polymer. The trehalose polymer is synthesized by dissolving azobisisobutyronitrile (AIBN) (5.28 mg, 3.22×10⁻² mmol) and styrenyl ether trehalose monomer (634 mg, 1.38 mmol) in a mixture of dimethylformamide (DMF) (2.31 mL) and H₂O (4.61 mL). Oxygen is removed by three cycles of freeze-pump-thaw and polymerization is initiated at 75° C. The polymerization is stopped after 8.5 h by immersing the vial into liquid nitrogen.

A third set of samples is vitrified in a vitrification medium including 600 mM Trehalose, 5 wt % Glycerol, 0.5 wt % Triton X-100 as per above, but also including an 8-arm polyethylene glycol bound to boronic acid. The polymer is synthesized dissolving an 8-arm PEG amine (400 mg, 10 kDa, 4×10⁻² mmol) and 4-formylphenylboronic acid (96 mg, 6.40×10⁻¹ mmol) in 2.8 ml of MeOH. NaBH₃CN (37.7 mg, 6.00×10⁻¹ mmol) is then added and stirred at 25° C. The remaining vitrification and storage protocols are identical.

Following storage, the tissue samples are reconstituted in lysis buffer and mRNA isolated as in Example 1. RNA is quantified by and analyzed by spectroscopy using a Take3 Plate of Synergy H1 Hybrid MF, BioTek Instrument. Briefly, 2 μl/well of each sample was added to the Take3 and ultra-pure RNase free water was used as a blank. The total RNA concentration was calculated based on the A260 reading using Gen5 software (1.0 is equivalent to ˜40 μg/ml ssRNA), and the A260/A280 ratio is used for RNA quality (A260/A280 ratio of 1.8-2.1+ is indicative of highly purified RNA). Results demonstrating robust yields of intact mRNA are illustrated in Table 2.

TABLE 2

| Sample | 260/280 ratio | ng/microliter | total yield (ng) |
|---|---|---|---|
| Liver | 2.168 | 97.12 | 6318 |
| Intestine | 2.099 | 108.48 | 7051 |

More robust results are expected using samples incubated with either the polyethylene glycol bound to boronic acid or the trehalose polymer.

### Example 4

When storing biological samples obtained from a non-sterile environment, the possibility of contamination from non-donor organism sources is always a possibility. To address this, a protocol was developed to selectively isolate the organism sample and exclude undesired bacterial contamination. A three-layer system was assembled with two layers of 8 micrometer nitrocellulose membrane that was either bound to mannose binding protein (MBL) or used as supplied, both on top of the PES membrane used for vitrification as in Example 1.

To test the ability of the assembled system to selectively isolate desired nucleic acid over bacterial nucleic acid, three samples of various bacterial strains was formed. Tested were E. coli, Staphylococcus epidermidis, and Pseudomonas aeruginosa. Each bacteria was diluted to 10⁴ colony forming units (CFU) in media. 100 microliters of the bacterial material was added to a pellet containing 10⁶ of each bacteria and the bacterial pellet resuspended. The resuspension was then added to the nitrocellulose membrane surface of the assembled three-layer membrane system and incubated for 30 minutes at room temperature. The three membranes were then separated and washed with PBS. The wash solution was then subjected to cell counting and plating on agar plates followed by culturing at 37° C. for two days. The results as the average from two experiments each performed in triplicate for E. coli are illustrated in Table 3.

TABLE 3

| Sample | % CFU | StDev | SEM |
|---|---|---|---|
| MBL coat | 35.69 | 11.47 | 8.11 |
| Neg. Ctl. | 93.08 | 2.34 | 1.66 |
| No Filter | 100 | 0 | 0 |

The results as the average from two experiments each performed in triplicate for S. epidermidis are illustrated in Table 4.

TABLE 4

| Sample | % CFU | StDev | SEM |
|---|---|---|---|
| MBL coat | 21.57 | 7.22 | 5.11 |
| Neg. Ctl. | 48.89 | 27.42 | 19.39 |
| No Filter | 100 | 0 | 0 |

The results indicate that the MBL bound filters selectively and robustly bound bacteria from the samples demonstrating that the system is capable of selectively removing bacterial contamination from an mRNA containing sample.

The cell counts in the PES membrane were also analyzed. The PES membrane was washed in PBS and the cells subjected to cell counting. The results as the average from two experiments each performed in triplicate are illustrated in Table 5.

TABLE 5

| | | Cells | | | % |
|---|---|---|---|---|---|
| Sample | Membrane | (*10⁵/ml) | StDev | SEM | Recovery |
| Jurkat + | MBL | 8.45 | 4.45 | 3.15 | 84.5 |
| Bacteria | Ctl | 8.8 | 2.55 | 1.8 | 88 |
| Jurkat | MBL | 8.85 | 0.21 | 0.15 | 88.5 |
| alone | Ctl | 9 | 0 | 0 | 90 |

In all instances, the PES membrane includes greater than 84% recovery of the cell originally loaded and the amount of Jurkat cells was independent of the presence or absence of MBL on the nitrocellulose layers. These results demonstrate a robust filtering system that is selective for bacterial cells and can be employed to selective storage of organism cells.

### FURTHER EXAMPLES

1. A process for storage of a biological sample, the process comprising:

providing a biological sample comprising one or more cells therein;

contacting the biological sample with a vitrification medium comprising a vitrification agent and a lysing agent to form a vitrification mixture;

vitrifying the vitrification mixture to generate a storage-stable sample.
2. The process according to further example 1, further comprising:

storing the storage-stable sample at a temperature of from greater than or equal to 16° C. to less than or equal to 30° C., optionally greater than 30° C., optionally greater than 50° C.
3. The process according to any of further examples 1-2, wherein the biological sample comprises nucleic acids, optionally RNA.
4. The process according to any of further examples 1-3, wherein the biological sample comprises whole blood, plasma, or serum.
5. The process according to further example 4, the biological sample comprising whole blood, serum, or plasma, wherein the vitrification mixture further comprises an anticoagulant.
6. The process according to any of further examples 1-5, wherein the vitrification mixture further comprises a buffering agent.
7. The process according to further examples 1-6, wherein the vitrification agent comprises dimethylsulfoxide, glycerol, sugars, polyalcohols, methylamines, betaines, antifreeze proteins, synthetic anti-nucleating agents, polyvinyl alcohol, cyclohexanetriols, cyclohexanediols, inorganic salts, organic salts, ionic liquids, or combinations thereof.
8. The process according to further example 7, wherein the vitrification agent comprises trehalose.
9. The process according to any one of further examples 1-8, wherein the lysing agent comprises a detergent.
10. The process according to further example 9, wherein the detergent comprises sodium dodecyl sulfate (SDS), 2-[4-(2,4,4-trimethylpentan-2-yl)phenoxy]ethanol (e.g., Triton X-100), CHAPS (3-[(3-cholamidopropyl)dimethylammonio]-1-propanesulfonate), guanidine hydrochloride, or combinations thereof.
11. The process according to any one of further examples 1-10, further comprising of enclosing said storage stable sample in a protective enclosure, said enclosure impervious to water and air, and storing said protective enclosure at a temperature between −196° C. to +60° C. for a storage time of 20 days or more.
12. The process according to any one of further examples 1-11, wherein vitrifying the vitrification mixture comprises desiccating the vitrification mixture above cryogenic temperature until the vitrification mixture enters into a glassy state.
13. The process according to further example 12, wherein the vitrification medium is within one or a plurality of capillary channels.
14. The process according to any one of further examples 1-13, wherein the vitrifying is performed for a desiccation time of from 1 second to 1 hour, optionally 10 minutes or less.
15. The process according to any one of further examples 1-14, further comprising incubating the vitrification mixture for greater than or equal to 5 minutes and less than or equal to 30 minutes prior to vitrification.
16. The process according to further example 15, wherein the incubating is at a temperature of greater than or equal to 18° C. to less than or equal to 37° C.
17. The process of any one of further example 1-16 wherein said biological sample is pretreated by contact with an isolation media that comprises one or more isolation agents therein.
18. The process of further example 17 wherein said isolation agent is a mannose binding lectin.
19. The process of further example 17 wherein said isolation media comprises nitrocellulose or polyvinylidene difluoride.
20. The process of any one of further examples 1-19, wherein said biological sample comprises a tissue and wherein said vitrification mixture further comprises a support material, optionally a polymer support material.
21. The process of further example 20 wherein said polymer is suitable for forming a hydrogel.
22. The process of further example 20 wherein said polymer is a polyethylene glycol.
23. The process of any one of further examples 20-22, wherein said vitrification medium further includes one or more attachment agents.
24. The process of further example 23, wherein the attachment agent is boronic acid.
25. The process of any one of further examples 20-24, wherein the support material is a switchable support material, the process further comprising subjecting said vitrification mixture to a stimuli to convert said switchable support material to a gel state.

Various aspects are disclosed herein; however, it is to be understood that the disclosed aspects are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the teachings disclosed herein. Furthermore, the terminology used herein is used only for the purpose of describing particular aspects of the present invention and is not intended to be limiting in any way.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Throughout this application, where publications are referenced, the disclosures of these publications in their entireties are hereby incorporated by reference into this application to more fully describe the state of the art to which this disclosure pertains.

While aspects of the invention have been illustrated and described, it is not intended that these aspects illustrate and describe all possible forms of the invention. Rather, the words used in the specification are words of description rather than limitation, and it is understood that various modifications and substitutions may be made thereto without departing from the spirit and scope of the invention.

1. A process for storage of a biological sample, the process comprising:
providing a biological sample comprising one or more cells therein;
contacting the biological sample with a vitrification medium comprising a vitrification agent and a lysing agent to form a vitrification mixture;
vitrifying the vitrification mixture to generate a storage-stable sample.

2. The process according to claim 1, further comprising:
storing the storage-stable sample at a temperature of from greater than or equal to 16° C. to less than or equal to 30° C., optionally greater than 30° C., optionally greater than 50° C.

3. The process according to claim 1, wherein the biological sample comprises nucleic acids.

4. The process according to claim 1, wherein the biological sample comprises whole blood, plasma, or serum.

5. The process according to claim 4, the biological sample comprising whole blood, wherein the vitrification mixture further comprises an anticoagulant.

6. The process according to claim 1, wherein the vitrification mixture further comprises a buffering agent.

7. The process according to claim 1, wherein the vitrification agent comprises dimethylsulfoxide, glycerol, a sugar, a polyalcohol, a methylamine, a betaine, antifreeze a protein, a synthetic anti-nucleating agent, polyvinyl alcohol, a cyclohexanetriol, a cyclohexanediol, an inorganic salt, an organic salt, an ionic liquid, or combinations thereof.

8. (canceled)

9. The process according to claim 1, wherein the lysing agent comprises a detergent, wherein the detergent is selected from the group consisting of sodium dodecyl sulfate (SDS), 2-[4-(2,4,4-trimethylpentan-2-yl)phenoxy]ethanol (e.g., Triton X-100), CHAPS (3-[(3-cholamidopropyl)dimethylammonio]-1-propanesulfonate), guanidine hydrochloride, or combinations thereof.

10. (canceled)

11. The process according to claim 1, further comprising of enclosing said storage stable sample in a protective enclosure, said enclosure impervious to water and air, and storing said protective enclosure at a temperature between −196° C. to +60° C. for a storage time of 20 days or more.

12. The process according to claim 1, wherein vitrifying the vitrification mixture comprises desiccating the vitrification mixture above cryogenic temperature until the vitrification mixture enters into a glassy state.

13. (canceled)

14. The process according to claim 12, wherein the vitrifying is performed for a desiccation time of from 1 second to 1 hour.

15. The process according to claim 1, further comprising incubating the vitrification mixture for greater than or equal to 5 minutes and less than or equal to 30 minutes prior to vitrification, or
wherein the incubating is at a temperature of greater than or equal to 18° C. to less than or equal to 37° C.

16. (canceled)

17. The process of claim 1, wherein said biological sample is pretreated by contact with an isolation media that comprises one or more isolation agents therein.

18. The process of claim 17 wherein said isolation agent is a mannose binding lectin.

19. The process of claim 17 wherein said isolation media comprises nitrocellulose or polyvinylidene difluoride.

20. A process of storing a tissue above cryogenic temperature comprising:
providing a biological tissue comprising one or more cells;
contacting the biological tissue with a vitrification medium comprising a vitrification agent and a support material, optionally a polymer support material, to form a vitrification mixture;
vitrifying the vitrification mixture to generate a storage-stable sample.

21. The process of claim 20 wherein said polymer may form a hydrogel, or wherein said polymer is a polyethylene glycol.

22. (canceled)

23. The process of claim 20, wherein said vitrification medium further includes one or more attachment agents.

24. The process of claim 23, wherein the attachment agent is boronic acid.

25. The process of claim 20, wherein the support material is a switchable support material, the process further comprising subjecting said vitrification mixture to a stimuli to convert said switchable support material to a gel state.

26-36. (canceled)
